(19) United States
(12) Patent Application Publication
(10) Pub. No.: US 2007/0297998 A1
(43) Pub. Date: Dec. 27, 2007

(54) Surface-Modified Zinc-Titanium Mixed Oxides

(75) Inventors: Jurgen Meyer, Stockstadt (DE); Steffen Hasenzahl, Morris Plains, NJ (US); Kai Schumacher, Hofheim (DE)

Correspondence Address: VENABLE LLP, P.O. BOX 34385, WASHINGTON, DC, 20043-9998, US

(73) Assignee: Degussa AG, Hanau (DE)

(21) Appl. No.: 11/630,240

(22) PCT Filed: Aug. 24, 2005
(86) PCT No.: PCT/EP05/09117

§ 371 Date: Jun. 1, 2007

(30) Foreign Application Priority Data
Sep. 23, 2004 (DE) 10 2004 046 093.0

(51) Int. Cl. A61K 8/19; A61P 17/18; C01B 33/04; C22C 14/00

(52) U.S. Cl. 424/59; 420/417; 423/347

(57) ABSTRACT

Surface-modified zinc-titanium mixed oxides are produced by treating zinc-titanium mixed oxides with a surface-modifying agent. They may be used for the production of sunscreen preparations.

The invention relates to surface-modified zinc-titanium mixed oxides, to a process for the production thereof and to the use thereof.

Cosmetic preparations, such as creams or lotions, containing UV filters, which are largely transparent on the skin and are pleasant to use, are used to protect the skin from excessively high intensity UV radiation.

The UV filters they contain comprise one or more organic compounds which absorb within the wavelength range between 290 and 400 nm: UVB radiation (290 to 320 nm), UVA radiation (320 to 400 nm).

The higher-energy UVB radiation causes the symptoms typical of sunburn and is also responsible for suppressing the immune defences, while UVA radiation, which penetrates into the deeper skin layers, causes premature skin ageing. Since the combined action of these two types of radiation is thought to promote the development of light-dependent skin conditions, such as for example skin cancer, the search has long been under way for possible ways of achieving a further significant improvement in the UV protection which has already been achieved.

It is known that microfine (ultrafine) pigments based on metal oxides can scatter, reflect and absorb UV radiation. They are accordingly an effective complement to the organic UV filters in sunscreen preparations.

Microfine titanium dioxide, for example, is accordingly widely used in cosmetic formulations because it is chemically inert and toxicologically safe and causes neither skin irritation nor sensitisation. In addition to titanium dioxide, microfine zinc oxide is also used.

Zinc oxide has long been widely used in pharmaceutically active skin preparations such as powders, ointments, creams and lotions. In cosmetic products, zinc oxide, like titanium dioxide, is used in decorative preparations due to its opacifying and brightening properties. Pigment grade zinc oxide has not gained widespread acceptance in sunscreen applications for the same reason as titanium dioxide pigment. Known zinc oxide pigment provides a white covering on surfaces. Zinc oxide has a relatively high refractive index of approx. 2.0. If transparent forms are to be obtained, micronised zinc oxide particles must be used, as is the case with titanium dioxide. Microfine zinc oxide generally has a particle size of 10 to 100 nm and a specific surface area of approx. 10 to 70 m²/g. Its action extends over the entire UV range, i.e. from UVA radiation via UVB radiation up to UVC. Zinc oxide with a relatively sharp UVA absorption edge at 370 nm absorbs better than titanium dioxide in the UVA range.

Particular problems arise if it is intended to use zinc oxide and titanium dioxide simultaneously in a sunscreen preparation. Such a combination is entirely sensible as zinc oxide absorbs more strongly in the UVA range and titanium dioxide more strongly in the UVB range, which would mean that broadband absorption could be achieved over the entire UV range. The two substances, however, have different isoelectric points: TiO₂ approx. 5 to 6 and ZnO approx. 9.5. At a pH value typical for cosmetic products of between 5 and 7, oppositely charged particles may be present which mutually attract one another and may give rise to agglomeration or flocculation. This risk primarily occurs when both metal oxides are present in the aqueous phase.

The object of the present invention was accordingly to overcome the existing disadvantages of combined use of titanium dioxide and zinc oxide and to provide a powder which combines the advantages of zinc oxide and titanium dioxide.

The present invention provides surface-modified zinc-titanium mixed oxides, characterised by the following physicochemical characteristics:

| | |
|---|---|
| BET surface area [m²/g]: | 1-120 |
| C content [%]: | 0.1-15 |
| ZnO content [%]: | 1-99* |
| TiO₂ content [%]: | 1-99* |

*relative to calcined substance

The present invention also provides a process for the production of surface-modified zinc-titanium mixed oxides, which process is characterised in that the zinc-titanium mixed oxides are treated with a surface-modifying agent.

Surface-modification may be performed by spraying the oxides with the surface-modifying agent at room temperature and then thermally treating the mixture at a temperature of 50 to 400° C. for a period of 1 to 6 hours.

An alternative method for surface-modifying the oxides may be performed by treating the oxides with the surface-modifying agent in vapour form and then thermally treating the mixture at a temperature of 50 to 800° C. for a period of 0.5 to 6 h. Thermal treatment may be performed under protective gas, such as for example nitrogen.

Surface modification may be performed continuously or batchwise in heatable mixers and dryers with sprayers. Suitable apparatuses may be, for example: plough bar mixers, disk dryers, fluidised or turbulent bed dryers.

The following silane compounds may be used as surface-modifying agents:
a) Organosilanes of the type (RO)₃Si(CₙH₂ₙ₊₁) and (RO)₃Si(CₙH₂ₙ₋₁)
   R=alkyl, such as for example methyl, ethyl, n-propyl, i-propyl, butyl
   n=1-20
b) Organosilanes of the type R′ₓ(RO)_{y}Si(CₙH₂ₙ₊₁) and
   R′x(RO)ySi(CₙH₂ₙ₋₁)
   R=alkyl, such as for example methyl, ethyl, n-propyl, i-propyl, butyl
   R′=alkyl, such as for example methyl, ethyl, n-propyl, i-propyl, butyl
   R′=cycloalkyl
   n=1-20
   x+y=3
   x=1.2
   y=1.2
c) Haloorganosilanes of the type X₃Si(CₙH₂ₙ₊₁) and
   X₃Si(CₙH₂ₙ₋₁)
   X=Cl, Br
   n=1-20
d) Haloorganosilanes of the type X₂(R′)Si(CₙH₂ₙ₊₁) and
   X₂(R′)Si(CₙH₂ₙ₋₁)
   X=Cl, Br
   R′=alkyl, such as for example methyl, ethyl, n-propyl, i-propyl, butyl
   R=cycloalkyl
   n=1-20
e) Haloorganosilanes of the type X(R′)₂Si(CₙH₂ₙ₊₁) and
   X(R′)₂Si(CₙH₂ₙ₋₁)
   X=Cl, Br
   R′=alkyl, such as for example methyl, ethyl, n-propyl, i-propyl, butyl
   R′=cycloalkyl
   n=1-20
f) organosilanes of the type (RO)₃Si(CH₂)ₘ—R′
   R=alkyl, such as methyl, ethyl, propyl
   m=0.1-20
   R′=methyl, aryl (for example —C₆H₅, substituted phenyl residues)
      —C₄F₉, OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
      —NR′R″R′″ (R′=alkyl, aryl; R″=H, alkyl, aryl; R′″=H, alkyl, aryl, benzyl, C₂H₄NR″″R′″″ with R″″=H, alkyl and R′″″=H, alkyl)
g) Organosilanes of the type (R″)ₓ(RO)_{y}Si(CH₂)ₘ—R′
   R=alkyl, such as for example methyl, ethyl, n-propyl, i-propyl, butyl
   R″=alkyl x+y=3
      =cycloalkyl x=1.2
         y=1.2
         m=0.1 to 20
   R′=methyl, aryl (for example —C₆H₅, substituted phenyl residues
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si (OR)₃
      —Sₓ—(CH₂)₃Si(OR)₃
      —SH
      NR′R″R′″ (R′=alkyl, aryl; R″=H, alkyl, aryl; R′″=H, alkyl, aryl, benzyl,
         C₂H₄NR″″R′″″ with R″″=H, alkyl and R′″″=H, alkyl)
h) Haloorganosilanes of the type X₃Si(CH₂)ₘ—R′
   X═Cl, Br
   m=0.1-20
   R′=methyl, aryl (for example —C₆H₅, substituted phenyl residues)
      —C₄F₉, —OCF₂—CHF—CF₃, —C₆F₁₃, —O—CF₂—CHF₂
      —NH₂, —N₃, —SCN, —CH═CH₂,
      —NH—CH₂—CH₂—NH₂
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
i) Haloorganosilanes of the type (R)X₂Si(CH₂)ₘ—R′
   X=Cl, Br
   R=alkyl, such as methyl, ethyl, propyl
   m=0.1-20
   R′=methyl, aryl (for example —C₆H₅, substituted phenyl residues)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃, wherein R may be methyl, ethyl, propyl, butyl
      —Sₓ—(CH₂)₃Si(OR)₃, wherein R may be methyl, ethyl, propyl, butyl
      —SH
j) Haloorganosilanes of the type (R)₂X Si(CH₂)ₘ—R′
   X=Cl, Br
   R=alkyl
   m=0.1-20
   R′=methyl, aryl (for example —C₆H₅, substituted phenyl residues)
      —C₄F₉, —OCF₂—CHF—CF₃, —C₆F₁₃, —O—CF₂—CHF₂
      —NH₂, —N₃, —SCN, —CH═CH₂, —NH—CH₂—CH₂—NH₂,
      —N—(CH₂—CH₂—NH₂)₂
      —OOC(CH₃)C═CH₂
      —OCH₂—CH(O)CH₂
      —NH—CO—N—CO—(CH₂)₅
      —NH—COO—CH₃, —NH—COO—CH₂—CH₃, —NH—(CH₂)₃Si(OR)₃
      —Sₓ—(CH₂)₃Si(OR)₃
      —SH
k) Silazanes of the type R′R₂Si—N(H)—SiR₂R′
   R=alkyl, vinyl, aryl
   R′=alkyl, vinyl, aryl
l) Cyclic polysiloxanes of the type D 3, D 4, D 5, wherein D 3, D 4 and D 5 are taken to mean cyclic polysiloxanes with 3, 4 or 5 units of the type —O—Si(CH₃)₂—.

For example Octamethylcyclotetrasiloxane=D 4 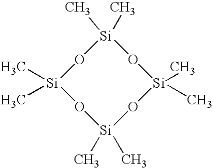
(m) Polysiloxanes or silicone oils of the type

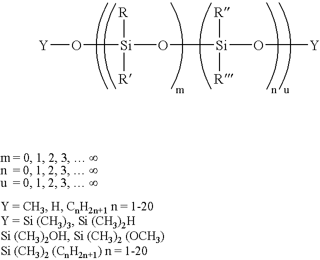

R=alkyl, such as CₙH₂ₙ₊₁, wherein n is 1 to 20, aryl, such as phenyl and substituted phenyl residues, (CH₂)ₙ—NH₂, H

R′=alkyl, such as CₙH₂ₙ₊₁, wherein n is 1 to 20, aryl, such as phenyl and substituted phenyl residues, (CH₂)ₙ—NH₂, H

R″=alkyl, such as CₙH₂ₙ₊₁, wherein n is 1 to 20, aryl, such as phenyl and substituted phenyl residues, (CH₂)ₙ—NH₂, H

R′″=alkyl, such as CₙH₂ₙ₊₁, wherein n is 1 to 20, aryl, such as phenyl and substituted phenyl residues, (CH₂)ₙ—NH₂, H

The following substances may preferably be used as surface-modifying agents:

octyltrimethoxysilane, octyltriethoxysilane, hexamethyldisilazane, 3-methacryloxypropyltrimethoxysilane, 3-methacryloxypropyltriethoxysilane, hexadecyltrimethoxy-silane, hexadecyltriethoxysilane, dimethylpolysiloxane, glycidyloxypropyltrimethoxysilane, glycidyloxypropyl-triethoxysilane, nonafluorohexyltrimethoxysilane, tridecafluorooctyltrimethoxysilane, tridecafluoro-octyltriethoxysilane, nonafluorohexyltriethoxysilane, aminopropyltriethoxysilane.

Octyltrimethoxysilane, octyltriethoxysilane, propyltrimethoxysilane, propyltriethoxysilane and dimethylpolysiloxanes may particularly preferably be used.

A powder mixture consisting of zinc-titanium mixed oxide particles, titanium oxide particles and zinc oxide particles may be used as the starting material, wherein the zinc-titanium mixed oxide particles have a composition according to the formula (ZnO)₁₋ₓ(TiO₂)ₓ, where 0.01<x<0.99, and are obtained from a thermal process and wherein the powder mixture exhibits remission which, in the UV range from 320 to 400 nm, is lower than that of titanium dioxide and, in the UV range below 320 nm, is lower than that of zinc oxide.

The titanium dioxide and zinc oxide particles may originate from thermal or pyrogenic processes, sol-gel processes or precipitation processes.

It is only the zinc-titanium mixed oxide particles for the purposes of the invention which originate from a thermal process.

A thermal process may, on the one hand, be taken to involve the conversion of zinc and titanium starting compounds at elevated temperatures. According to the invention, thermal processes may, on the other hand, be taken to comprise pyrogenic processes with subsequent thermal treatment of the reaction mixture. A pyrogenic process may be taken to comprise flame hydrolysis or flame oxidation of metal or metalloid compounds in the gas phase in a flame, produced by the reaction of a fuel gas, preferably hydrogen, and oxygen. In such a process, highly disperse primary particles are initially formed which, as the reaction proceeds, may combine to form aggregates and the latter may in turn further congregate to form agglomerates. The BET surface area of these primary particles may generally have a value of between 5 and 600 m²/g.

It is known to produce zinc-titanium mixed oxide by a pyrogenic process as described in EP-A-1138632. It has, however, been found that, at the desired high zinc oxide contents (>20 wt. %), a non-homogeneous product mixture is obtained which is unsuitable for cosmetic purposes. The product produced according to EP-A-1138632 with a zinc oxide content of approx. 20 wt. % thus cannot be used for the purposes of the invention.

It is an essential feature of the invention that, in order to achieve remission which, in the UV range from 320 to 400 nm, is lower than that of titanium dioxide and, in the UV range below 320 nm, is lower than that of zinc oxide, the zinc-titanium mixed oxide particles originate from a thermal process.

The powder mixture according to the invention may still contain small quantities or contaminants which are introduced by the starting materials and/or by process contaminants. These amount to less than 1 wt. %, even in general to less than 0.1 wt. %.

In a preferred embodiment of the invention, the content of the zinc-titanium mixed oxide particles in the powder mixture may amount to at least 50 wt. %. A zinc-titanium mixed oxide content of at least 80 wt. % may be particularly preferred.

The zinc-titanium mixed oxide particles may preferably have a composition (ZnO)₁₋ₓ(TiO₂)ₓ, where 0.05<x<0.80.

The zinc-titanium-mixed oxide particles may be amorphous or crystalline. Crystalline zinc-titanium mixed oxide particles may be preferred for the purposes of the invention. Crystalline means that defined reflections are observable in an X-ray diffractogram, the width of which is determined by the size of the primary particles.

It may furthermore be advantageous if the isoelectric point of the powder mixture usable according to the invention is between that of zinc oxide and that of titanium dioxide. The isoelectric point of zinc oxide is at approx. 9.2, that of titanium dioxide at approx. 5 to 6.

The titanium dioxide particles of the powder mixture usable according to the invention may comprise rutile, anatase and brookite modifications, the ratio of which to one another is not limited. Preferably, however, the proportion of the rutile modification of the titanium dioxide particles of the powder mixture usable according to the invention may amount to at least 1%, relative to the sum of rutile and anatase modification.

In a preferred embodiment, the powder mixture usable according to the invention may have a BET surface area which is between 1 and 100 m²/g. The range may particularly preferably be between 5 and 40 m²/g. The BET surface area is determined according to DIN 66131.

The chlorine content of the powder mixture usable according to the invention may, if desired, be less than 500 ppm. In particular embodiments it may be less than 100 ppm.

There are two process for the production of the powder mixture usable according to the invention.

The first process is performed by homogeneously mixing an aerosol, which contains a zinc compound, with a mixture containing a titanium compound, optionally an inert gas, a fuel gas and a gas containing free oxygen in a mixing chamber of a burner as is used for the production of pyrogenic oxides, igniting the mixture of all the components at the mouth of the burner and combusting it in a cooled flame tube, then separating the resultant solids from the gaseous reaction products, optionally purifying them, and thermally treating them.

The composition of the powder mixture may be varied by modifying flame parameters and the thermal post-treatment.

The zinc and titanium compound may preferably be present in a ratio such that the powder mixture usable according to the invention contains between 20 and 95 wt. % of zinc oxide.

Titanium tetrachloride may preferably be used as the titanium compound.

The aerosol may preferably be produced by atomisation by means of a two-fluid nozzle or by an aerosol generator. The second process for the production of the powder mixture usable according to the invention is performed by dispersing a titanium dioxide powder in the presence of a solution of a zinc compound, wherein the ratio of titanium dioxide and zinc salt corresponds to the subsequently desired ratio of titanium dioxide and zinc oxide in the final product, the mixed oxide particles being calculated separately as titanium dioxide and zinc oxide, then removing the solvent by evaporation and thermally treating the residue.

In both processes, thermal treatment may preferably proceed at temperatures of 400 to 600° C. over a period of 0.5 to 8 hours.

Selection of the zinc salts is not restricted in either of the processes. For example, zinc chloride, zinc nitrate and/or organozinc compounds, such as for example zinc acetate, may be used.

Dissolution of the zinc compound may proceed in an aqueous or organic solvent. An aqueous solution is preferred.

Pyrogenically produced titanium dioxide powder, for example titanium dioxide P 25 from Degussa, may preferably be used as the titanium dioxide powder.

The present invention also provides a sunscreen preparation which contains the powder mixture according to the invention in a proportion of between 0.01 and 25 wt. %. The sunscreen preparation according to the invention may additionally be used in mixtures with known inorganic UV-absorbing pigments and/or chemical UV filters.

Titanium dioxides, zinc oxides, aluminium oxides, iron oxides, cerium oxides, zirconium oxides, barium sulfate or mixtures thereof may be used as known UV-absorbing pigments.

Chemical UV filters which may be used are any water- or oil-soluble UVA and UVB filters known to the person skilled in the art, the following being mentioned by way of non-limiting examples: sulfonic acid derivatives of benzophenones and benzimidazoles, derivatives of dibenzoylmethane, benzylidene camphor and the derivatives thereof, derivatives of cinnamic acid and the esters thereof, or esters of salicylic acid. Selected examples may be: 2-hydroxy-4-methoxybenzophenone, 2-hydroxy-4-methoxybenzophenone 5-sulfonic acid, 2-hydroxy-4-methoxybenzophenone 5-sulfonate sodium salt, dihydroxydimethoxybenzophenone, dihydroxydimethoxy-benzophenone sulfonate sodium salt, tetrahydroxybenzo-phenone, p-aminobenzoic acid, ethyl p-aminobenzoate, glyceryl p-aminobenzoate, amyl p-dimethylaminobenzoate, octyl p-dimethylaminobenzoate, ethyl p-methoxycinnamate, isopropyl p-methoxycinnamic acid ester, octyl p-methoxy-cinnamic acid ester, 2-ethylhexyl-p-methoxycinnamic acid ester, p-methoxycinnamic acid ester sodium salt, glyceryl di-p-methoxycinnamic acid ester mono-2-ethylhexanoate, octyl salicylate, phenyl salicylate, homomenthyl salicylate, dipropylene glycol salicylate, ethylene glycol salicylate, myristyl salicylate, methyl salicylate, 4-t-butyl-4-methoxydibenzoylmethane and 2-(2′-hydroxy-5′-methylphenyl)benzotriazole.

Among these, 2-ethylhexyl-p-methoxycinnamic acid ester and 4-tert.-butyl-4′-methoxydibenzoylmethane are preferred by virtue of the UV protection they provide and their skin compatibility.

The sunscreen preparation according to the invention may contain solvents such as water, mono- or polyhydric alcohols, cosmetic oils, emulsifiers, stabilisers, consistency regulators such as carbomers, cellulose derivatives, xanthan gum, waxes, bentones, pyrogenic silicas and further substances conventional in cosmetics such as vitamins, antioxidants, preservatives, dyes and fragrances.

The sunscreen preparation according to the invention may typically assume the form of an emulsion (O/W, W/O or multiple), aqueous or aqueous-alcoholic gel or oil gel and be produced in the form of lotions, creams, milk sprays, foam, sticks or other usual forms.

The general preparation of sunscreen preparations is furthermore described in A. Domsch, “Die kosmetischen Präparate”, Verlag für chemische Industrie (ed. H. Ziolkowsky), 4th edition, 1992 or N. J. Lowe and N. A. Shaat, Sunscreens, Development, Evaluation and Regulatory Aspects, Marcel Dekker Inc., 1990.

The present invention also provides the use of the powder mixture according to the invention as an adsorbent for UV radiation.

The surface-modified zinc-titanium mixed oxides according to the invention exhibit the following advantages:

In sunscreen formulations, the zinc-titanium mixed oxides according to the invention exhibit a higher sun protection factor and a broadband protective action in the UVA and UVB range. In comparison with titanium dioxide and zinc oxide as individual components, they may moreover be substantially more readily formulated. They furthermore bring about a lower viscosity. They thus have a reduced thickening action and so yield sunscreen preparations which can be applied better.

### EXAMPLES

Production of the Oxides

Remission is determined by means of a Perkin-Elmer model P554 spectrometer with remission sphere.

BET surface area is determined according to DIN 66131.

### Example 1

0.60 kg/h of TiCl₄ are volatilised in a vaporiser at approx. 150° C. and the vapour is passed into the mixing chamber of a burner by means of 0.14 Nm³/h of nitrogen. In this chamber, the stream of gas is mixed with 1.4 Nm³/h of hydrogen and 2.0 Nm³/h of dried air and supplied to the flame through the mouth of the burner.

The burner consists of two concentric tubes, in the middle of which there is additionally located a two-fluid nozzle for atomising liquids by means of a gas stream, said nozzle ending at the level of the burner mouth.

0.2 Nm³/h of hydrogen are passed as jacket gas through the outer tube of the burner. 330 ml/h of an aqueous zinc acetate solution (400 g/l) are pumped by means of a gear pump through the liquid tube of the two-fluid nozzle (internal diameter 0.2 mm), the solution being atomised by means of 550 l/h of air. The gases and atomised liquid are combusted in the reaction chamber and cooled to approx. 110° C. in a downstream coagulation section. The resultant powder is then deposited in a filter. The powder mixture usable according to the invention is obtained in a subsequent heat treatment step at 600° C. for a duration of 40 minutes.

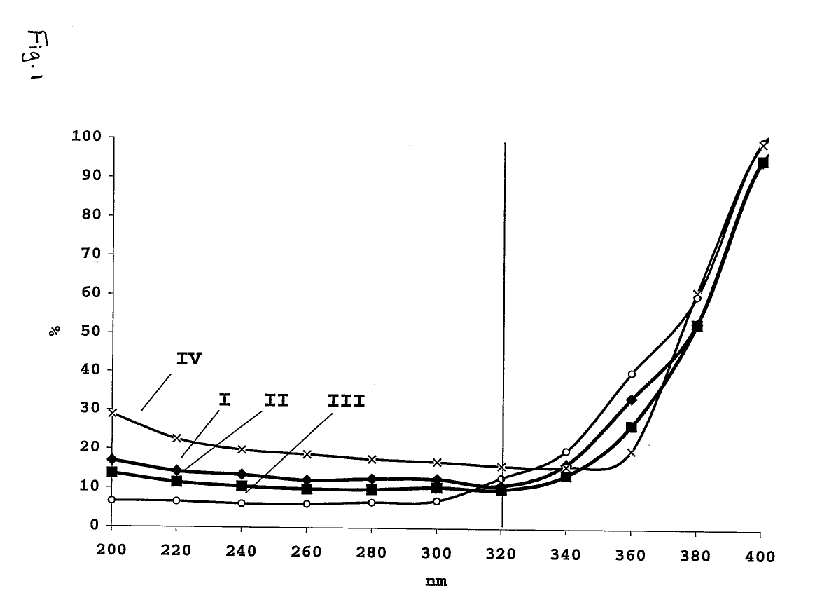

X-ray diffraction analysis of the powder mixture before heat treatment reveals that it comprises a mixture of titanium dioxide and zinc oxychloride (Zn₂OCl₂). X-ray diffraction analysis after heat treatment reveals a mixture of zinc-titanium mixed oxide, titanium dioxide, zinc oxide with a BET surface area of 40 m²/g, a pH value (4% aqueous dispersion) of 6.45, a bulk density of 290 g/l and a tamped density of 340 g/l. FIG. 1 shows the remission of this powder mixture.

The titanium dioxide exhibits a rutile/anatase ratio of 30:70 before heat treatment and of 45:55 after heat treatment.

### Example 2A

Pyrogenically produced titanium dioxide (P25, Degussa) is dispersed by means of a laboratory stirrer in a zinc nitrate solution in 100 ml of water. The water is then removed at 90° C. and the residue treated at 550° C. over a period of 3 hours. Heat treatment at 550° C. is then performed for 3 hours.

Table 1 shows the quantities used and the physicochemical properties obtained in Examples 2A-D. The stated values for TiO₂ and ZnO were determined by X-ray fluorescence analysis and include zinc-titanium mixed oxide. X-ray diffraction analysis reveals the presence of a mixture of zinc-titanium mixed oxide, titanium dioxide and zinc oxide.

FIG. 1 shows the remission (in %) of the powder from Example 1 (denoted I) and Example 2C (II) in comparison with a pyrogenically produced titanium dioxide (P25, Degussa, III) and a zinc oxide (Nanox 100, Elementis, IV) as a function of wavelength.

The powder mixtures (I) and (II) usable according to the invention exhibit remission which, in the UV range from 320 to 400 nm, is lower than that of titanium dioxide and, in the UV range below 320 nm, is lower than that of zinc oxide.

TABLE 1
Quantities used and physicochemical properties of
Examples 2A-D

| | Starting substances | | | Product | | |
|---|---|---|---|---|---|---|
| | TiO₂ | Zinc salt | | TiO₂ | ZnO | BET |
| Example | g | | g | [wt. %] | [wt. %] | [m²/g] |
| 2-A | 10 | ZnCl₂ | 5 | 87.7 | 12.3 | 25 |
| 2-B | 5 | ZnNO₃⁽*⁾ | 10 | 68.8 | 31.2 | 19 |
| 2-C | 5 | ZnNO₃⁽*⁾ | 30 | 33.5 | 66.5 | 14 |
| 2-D | 5 | ZnNO₃⁽*⁾ | 50 | 22.6 | 77.4 | 8 |

⁽*⁾as hexahydrate

The powder mixture from Example 2-C has an isoelectric point of 8.3.

Production of the Products

The oxides, or the powder mixture according to Table 1, are introduced into a mixer for surface modification and, while being vigorously mixed, are sprayed, optionally initially with water and then with the surface-modifying agent.

Once spraying is complete, mixing may be continued for a further 15 to 30 minutes and heat treatment then performed at 50 to 400° C. for 1 to 4 hours. The water used may be acidified to a pH value of 7 to 1 with an acid, for example hydrochloric acid. The silanising agent used may be dissolved in a solvent, such ethanol for example.

TABLE 2
Production of the surface-modified oxides

| | | | Parts | Parts | Heat | |
|---|---|---|---|---|---|---|
| | | | SMA*/ | H₂O | treatment | Heat |
| | | Surface- | 100 | 100 | temper- | treatment |
| | | modifying | parts | parts | ature | time |
| Example | Oxide | agent | oxide | oxide | [° C.] | [h] |
| 1 | 2-A | A | 2 | — | 120 | 2 |
| 2 | 2-A | A | 2 | 0.2 | 120 | 2 |
| 3 | 2-A | A | 2 | 0.2** | 120 | 2 |
| 4 | 2-A | B | 3 | — | 120 | 2 |
| 5 | 2-A | C | 2.5 | — | 350 | 2 |
| 6 | 2-B | A | 1.5 | — | 120 | 2 |
| 7 | 2-B | C | 2 | — | 350 | 2 |
| 8 | 2-C | A | 1.5 | — | 120 | 2 |
| 9 | 2-C | B | 2 | — | 120 | 2 |
| 10 | 2-C | C | 2 | — | 350 | 2 |
| 11 | 2-D | A | 1 | — | 120 | 2 |
| 12 | 2-D | C | 1.5 | — | 350 | 2 |

*SMA = surface-modifying agent
**0.001 n HCl was used instead of H₂O
Surface-modifying agent:
A = octyltrimethoxysilane
B = hexadecyltrimethoxysilane
C = dimethylpolysiloxane

TABLE 3
Physicochemical data of the surface-modified oxides

| | BET specific surface | |
|---|---|---|
| Example | area [m²/g] | C content [%] |
| 1 | 23 | 0.6 |
| 2 | 22 | 0.7 |
| 3 | 22 | 0.7 |
| 4 | 22 | 0.9 |
| 5 | 22 | 0.5 |
| 6 | 17 | 0.5 |
| 7 | 17 | 0.4 |
| 8 | 13 | 0.4 |
| 9 | 12 | 0.5 |
| 10 | 12 | 0.4 |
| 11 | 7 | 0.2 |
| 12 | 7 | 0.2 |

Production of Sunscreen Formulations

### EXAMPLES

FORMULATION 1
W/O sunscreen Formulation with SPF 4-10

| Phase | Constituents | INCI name | % |
|---|---|---|---|
| A | Isolan GI 34 (Degussa, | Polyglyceryl-3 | 3.00 |
| | Goldsclimidt) | isostearate | |
| | Ricinus Oil | Ricinus communis | 1.20 |
| | Tegosoft Liquid (Degussa, | Cetearyl ethylhexanoate | 5.00 |
| | Goldschmidt) | | |
| | Glycerol 86% | Glycerin | 3.00 |
| B | Paracera W 80 | | 1.80 |
| | Isohexadecane | Isohexadecane | 5.00 |
| C | Micropiginent from | Titanium dioxide and/or | 4.00 |
| | Examples 6-10 or | zinc oxide | |
| | Comparative Examples 1-3 | | |
| D | Magnesium sulfate | | 0.5 |
| | Demineralised water | Water | 66.5 |

The constituents of phase A are mixed and heated 70° C. while being stirred. Phase B is melted at 80° C. and added to phase A with stirring. The micropigment (phase C) is added, likewise with stirring, and dispersed for five minutes with an Ultra-Turrax mixer. Phase D, which has been heated to 70° C., is then added to the combined phases A, B and C with stirring, and this mixture is homogenised for a further 10 minutes with an Ultra-Turrax mixer. The composition is allowed to cool to room temperature while being stirred.

FORMULATION 2
W/O sunscreen formulation with elevated SPF

| Phase | Constituent | INCI name | % |
|---|---|---|---|
| A | ABIL ® EM 90 (Degussa, | Cetyl PEG/PPG-10/1 | 3.00 |
| | Goldschmidt) | dimethicone | |
| | Isohexadecane (BASF) | Isohexadecane | 6.25 |
| | Cosmacol (Condea Chemie, | Tridecyl salicylate | |
| | Germany) | | |
| | Uvinul ® MC 80 (BASF) | Ethylhexyl | 8.50 |
| | | methoxycinnamate | |
| | Uvinul ® N 539 (BASF) | Octocrylen | 2.00 |
| | Uvinul ® NBC 95 (BASF) | 4-Methylbenzylidene | 2.00 |
| | | camphor | |
| | Vitamin E acetate (BASF) | Tocopheryl acetate | 1.00 |
| B | Micropigment from | Titanium dioxide and/or | 5.00 |
| | Examples 6-10 or | zinc oxide | |
| | Comparative Examples 1-3 | | |
| C | Glycerol 87% (BASF) | Glycerin | 4.00 |
| | D-Panthenol 75 W (BASF) | Panthenol | 1.50 |
| | Sodium ascorbyl phosphate | Sodium ascorbyl phosphate | 0.10 |
| | (BASF) | | |
| | Sodium chloride (BASF) | Sodium chloride | 1.00 |
| | Demineralised water | Water | 55.65 |

Method: The constituents of phase A are mixed and heated to approx. 60° C. The micropigment (phase B) is added with stirring and dispersed for five minutes (Ultra-Turrax). Phase C is then stirred into the combined phases A and B and homogenised for 10 minutes with an Ultra-Turrax mixer. The composition is allowed to cool to room temperature while being stirred.

Micropigments used for formulations 1 and 2

| | | | Behaviour |
|---|---|---|---|
| | | Micropigment | towards |
| Micropigment | INCI name | content | water |
| Example 6 | Titanium dioxide | approx. 68 wt. % | hydro- |
| | (and) zinc oxide | TiO₂ and approx. | phobic |
| | (and) trimethoxy- | 31 wt. % ZnO | |
| | caprylsilane | | |
| Example 7 | Titanium dioxide | approx. 68 wt. % | hydro- |
| | (and) zinc oxides | TiO₂ and approx. | phobic |
| | (and) dimethicone | 31 wt. % ZnO | |
| Comparative | Trimethoxycaprylsi- | approx. 95 wt. % | hydro- |
| Example 1 | lane (and) titanium | TiO₂ | phobic |
| AEROXIDE ® | dioxide | | |
| TiO₂ T | | | |
| 805 (Degussa) | | | |
| Comparative | Titanium dioxide | approx. 77 wt. % | hydro- |
| Example 2 | (and) alumina | ZnO | phobic |
| UV-Titan M160 | (and) stearic acid | | |
| Example 8 | Zinc oxide (and) | approx. 33 wt. % | hydro- |
| | titanium dioxide | TiO₂ and approx. | phobic |
| | (and) trimethoxy- | 66.0 wt. % ZnO | |
| | caprylsilane | | |
| Example 9 | Zinc oxide (and) | approx. 33 wt. % | hydro- |
| | titanium dioxide | TiO₂ and approx. | phobic |
| | (and) trimethoxy- | 66.0 wt. % ZnO | |
| | cetylsilane | | |
| Example 10 | Zinc oxide (and) | approx. 33 wt. % | hydro- |
| | titanium dioxide | TiO₂ and approx. | phobic |
| | (and) dimethicone | 66.0 wt. % ZnO | |
| Comparative | Zinc oxide (and) | approx. 98 wt. % | hydro- |
| Example 3 | dimethicone | ZnO | phobic |
| Z-COTE HP1 | | | |
| (BASF) | | | |

Characterisation of Sunscreen Formulations

All the formulations were stable after three months storage at 45° C., no phase separation having occurred.

In vitro sun protection factors were determined using the method of Tronnier and Kockott (Kockott, D.: In vitro evaluation of sunscreen preparations, Kosmetische Medizin 19, 290-293 (1998); Tronnier H., Kockott D., Meick, B. Hani, N., Heinrich, U., Parfumerie und Kosmetik 5, 326-329 (1996)). To this end, the formulations were applied in a film thickness of 1 mg/cm² onto a roughened polymethyl methacrylate microscope slide. The sample was left to stand for 15 minutes in air and then irradiated with a 180 W xenon lamp with reflector for a total of 12 minutes. The measurement was repeated twice in total and the average stated.

Viscosity was determined with a Brookfield Rheometer (spindle RDV VII+; 10 min⁻¹).

Results

| | in vitro | | Viscosity | |
|---|---|---|---|---|
| | SPF | | (mPa · s) | |
| | Formu- | Formu- | Formu- | Formu- |
| Micropigment | lation 1 | lation 2 | lation 1 | lation 2 |
| Example 6 | 10.1 | 28.3 | 15,600 | 22,000 |
| Example 7 | 9.3 | 27.9 | 14,900 | 22,000 |
| Comparative | 8.2 | 25.1 | 17,300 | 24,000 |
| Example 1 | | | | |
| AEROXIDE ® | | | | |
| TiO₂ T | | | | |
| 805 (Degussa) | | | | |
| Comparative | 8.0 | 25.0 | 18,000 | 25,000 |
| Example 2 | | | | |
| UV-Titan M160 | | | | |
| Example 8 | 6.3 | 22.2 | 14,900 | 21,000 |
| Example 9 | 6.1 | 23.7 | 15,000 | 21,500 |
| Example 10 | 6.5 | 24.1 | 16,100 | 20,900 |
| Comparative | 4.1 | 20.1 | 18,900 | 24,500 |
| Example 3 | | | | |
| Z-COTE HP1 | | | | |
| (BASF) | | | | |

The investigations show that the titanium-zinc mixed oxides with an elevated titanium dioxide content (Examples 6 and 7) yield formulations with a higher in vitro SPF than the commercial titanium dioxide UV filter (Comparative Examples 1 and 2). The formulations with the titanium-zinc mixed oxides with an elevated zinc oxide content (Examples 8 to 10) likewise exhibit a higher in vitro SPF than the formulation produced with the commercial Comparative Example 3.

Moreover, all the formulations with micropigments from Examples 6 to 10 have a lower viscosity than the formulations produced with the Comparative Examples. The micropigments according to the invention accordingly thicken the formulations less than the commercial Comparative Examples and so yield sunscreen preparations which can be applied better.

1. Surface-modified calcined zinc-titanium mixed oxides having a composition according to the formula (ZnO)₁₋ₓ(TiO₂)ₓ, where 0.01<x<0.99, and having
BET surface area (m²/g) between 1-120 and
C content between 0.1-15.

2. A process for the production of the surface-modified calcined zinc-titanium mixed oxides according to claim 1, comprising treating calcined zinc-titanium mixed oxides having a composition according to the formula (ZnO)₁₋ₓ(TiO₂)ₓ, where 0.01<x<0.99, with a silane containing surface-modifying agent to form a modified surface.

3. An adsorbent composition for UV radiation comprising surface-modified calcined zinc-titanium mixed oxides having a composition according to the formula (ZnO)₁₋ₓ(TiO₂)ₓ, where 0.01<x<0.99.

4. A sunscreen preparation, the surface-modified calcined zinc-titanium mixed oxides according to claim 1.
